(19) United States
(12) Patent Application Publication
(10) Pub. No.: US 2005/0163714 A1
(43) Pub. Date: Jul. 28, 2005

(54) Capsules of multilayered neutral polymer films associated by hydrogen bonding

(76) Inventors: Svetlana A. Sukhishvili, Maplewood, NJ (US); Veronika Kozlovskaya, Jersey City, NJ (US); Eugenia Kharlampieva, Jersey City, NJ (US)

Correspondence Address: DOCKET ADMINISTRATOR;LOWENSTEIN SANDLER PC, 65 LIVINGSTON AVENUE, ROSELAND, NJ, 07068, US

(21) Appl. No.: 10/954,922

(22) Filed: Sep. 30, 2004

Related U.S. Application Data
(60) Provisional application No. 60/508,754, filed on Oct. 2, 2003.
(60) Provisional application No. 60/510,449, filed on Oct. 10, 2003.

(51) Int. Cl. A61K 48/00; A61K 49/00; A01N 25/00; A61K 9/48; A61K 9/14

(52) U.S. Cl. 424/9.6; 424/451; 424/489; 514/44; 424/405

(57) ABSTRACT

Micro-, and nano-scale capsules comprising neutral (uncharged) polymeric layers, layers associated by hydrogen bonding and methods for making such capsules. The capsules of the invention are layered upon a core particle using a layer-by layer-technique. The capsule walls of the capsules of the invention give a tailored response to external stimuli.

This application claims the benefit of U.S. Provisional Application No. 60/508,754, filed Oct. 2, 2003, entitled Hydrogen-Bonded Polymer Capsules Formed by Layer-by-Layer Self-Assembly, by S. Sukhishvili et al., which application is hereby incorporated herein by reference in its entirety; and 60/510,449, filed Oct. 10, 2003, entitled Capsules of Multilayered Neutral Polymer Films Associated by Hydrogen Bonding, by S. Sukhishvili et al., which application is hereby incorporated herein by reference in its entirety.

### 1. FIELD

The invention is directed to capsules of multilayered neutral polymer films, wherein the uncharged layers are associated by hydrogen bonding. Particularly, the invention is directed to micro- and nano-sized capsules.

### 2. BACKGROUND

In many applications, it is desirable to controllably release substances. In recent years, micro-capsules have received considerable attention for controlled release of encapsulated active ingredients, particularly, in the fields of biotechnology; medicine; pharmaceuticals, such as drug delivery; foods; agriculture; perfumery; personal care; and cosmetics. See e.g., K. PARK, CONTROLLED DRUG DELIVERY: CHALLENGES AND STRATEGIES, (Am. Chem. Soc., Washington D.C., 1997); J. Kost, R. Langer, Responsive Polymeric Delivery Systems, 46 ADVANCED DRUG DELIVERY REVIEWS 125 (2001).

Capsules comprised of electrostatically associated polymeric multilayers have been researched for use in controlled delivery, See e.g., G. B. Sukhorukov, et al., pH Controlled Macromolecule Encapsulation In And Release From Polyelectrolyte Multilayer Nanocapsules, 22 MACROMOL. RAPID COMMUN., 44 (2001). Such electrostatically associated multilayered capsules are typically prepared by layer-by-layer sequential adsorption of electrostatically charged polymers on a substrate. G. Decher & J.-D. Hong, Buildup of Uultrathin Multilayer Films by a Self-assembly Process: I. Consecutive Adsorption of Anionic and Cationic Bipolar 46 MACROMOL. CHEM. MACROMOL. SYMP. 321 (1991); P. Fisher et al., Polyelectrolytes Bearing Azobenzenes for the Functionalization of Multilayers, 137 MACROMOL. SYMP. 1 (1999). This self-assembly technique relies on the interlayer attraction between alternating polymer layers due to alternating positive and negative electrostatic charge. Ibid. Using this technique, ultrathin electrostatically charged films have been deposited onto micro- and nano-sized particulate substrates.

Advantageously, if the multilayer is deposited on a soluble particulate substrate, the substrate can subsequently be dissolved under appropriate conditions to produce hollow electrostatically associated multilayered polymer film capsules. See e.g., G. B. Sukhorukov, et al., Stepwise Polyelectrolyte Assembly on Particle Surfaces: a Novel Approach to Colloid Design, 9 POLYM. ADV. TECHNOL. 759 (1998). Such capsules encapsulating dyes, small organic molecules, enzymes, and biological macromolecules have been produced. See e.g., A. A Antipov, et al., Sustained Release Properties of Polyelectrolyte Multilayer Capsule, 105 PHYS. CHEM. B 2281 (2001); X. Qiu, et al., Permeability of Ibuprofen in Various Polyelectrolyte Multilayer, 286 MATER. ENG. 591 (2001); F Caruso, et al., Microencapsulation of Uncharged Low Molecular Weight Organic Materials by Polyelectrolyte Multilayer Self-Assembly, 16 LANGMUIR 8932 (2000); F. Caruso, et al., Enzyme Encapsulation in Layer-by-Layer Engineered Polymer Multilayer Capsules, 16 LANGMUIR 1485 (2000); G. B. Sukhorukov, et al., pH-Controlled Macromolecule Encapsulation in and Release from Polyelectrolyte Multilayer Nanocapsules, 22 RAPID COMMUN. 44 (2001).

But unfortunately, it is difficult to fine-tune the electrostatically associated multilayered capsule systems to the particular controlled-delivery application. Therefore, such capsules have limited utility for controlled release of substances into the surrounding environment.

What is needed is a multi-layered micro- and nano-sized capsule system that permits flexibility of design so that the capsules can be tailored to controllably encapsulate and/or release a substance depending on the particular application and environment. Such capsules would be useful in a wide range of applications, such as in drug-delivery and other controlled-delivery applications.

### 3. SUMMARY

The present invention provides capsules comprising neutral (uncharged) layers of polymers that are associated by hydrogen bonding (as opposed to electrostatic charge). Preferably, the capsules of the invention are millimeter, micrometer, or nanometer-scale capsules, more preferably, nanometer-scale capsules.

The invention also provides methods for making such capsules. The capsules of the invention are prepared by layering the neutral polymer films upon a core particle using a layer-by layer-technique. Thus, in one embodiment, the capsules of the invention comprise a core particle.

The capsules of the invention are useful to deliver the core particle or other encapsulated substance in a controlled and well-defined manner upon exposure to a particular external stimuli, such as a change in pH, salt concentration, temperature, solvent composition, application of an electric field, exposure to sunlight, or other external environmental change, depending on the specific composition of the capsules.

In contrast to electrostatic self-assembly previously used to produce multilayer capsules, the hydrogen-bonding interactions of the invention represent an advantageous alternative driving force for the layer-by-layer growth of multilayer capsules.

A hydrogen bond is a relatively weak secondary interaction between: (1) a hydrogen atom bound to a more electronegative atom; and (2) another atom that is also more electronegative than hydrogen and that has one or more lone electron pairs, such as oxygen, sulfur, nitrogen, or phosphorous. Hydrogen bonding has been extensively studied. See e.g., F. ALBERT COTTON & GEOFFREY WILKINSON, ADVANCED INORGANIC CHEMISTRY 90-94 (5th ed., 1988), hereby incorporated herein by reference.

In one embodiment, the capsules of the invention give a tailored response to external stimuli. For example, in one version of this embodiment, the capsules are sensitive to the external pH value. The capsules can be designed to release the core particle or encapsulated substance in response to specific external stimuli. For example, the capsule walls can be designed to release the core particle or other encapsulated substance at a selected pH, over a period of time, depending of the layer number and the polymer system. The pH at which the capsules of the invention begin a steep increase in the release rate of the core particle or the encapsulated substance is referred to herein as the critical pH. The critical pH value is controlled by the choice of polymer system and other variables.

In another embodiment, the hydrogen-bonded multilayers capsules of the invention demonstrate remarkable stability at low pH (e.g., pH about 1 or less), in contrast to the known electrostatically associated polymer multilayers, specifically those composed of a weak polyacids, which rapidly and uncontrollably dissociate under acidic conditions.

In certain embodiments, the intermolecular hydrogen-bond induced adhesion between the layers of the capsules of the invention decreases and they begin to disintegrate. In one embodiment, however, the hydrogen-bonded multilayers of the capsules of the invention are stabilized at neutral and basic pH values by covalent cross-linking. If the polymeric layers of the capsules of the invention are cross-linked, the multilayer wall acquires increased stability, even at high pH values. In certain aspects of this embodiment, the cross-linked wall is an ultrathin gel whose thickness and properties can be conveniently controlled by the number of polymer layers initially deposited onto a solid core, the degree of cross-linking, and the choice of the polymer system.

In yet another embodiment of the invention, the capsules of the invention comprise an encapsulated substance, such as a pharmaceutical or other bioactive material. Such capsules can be used to deliver the encapsulated substance in a controlled manner upon inserting them in an environment where the capsule wall is designed to release the substance. For example, the capsules of the invention can be tailored to controllably disintegrate in the human body at a particular pH value. Different areas of the human body have different pH values, for example, the pH of the alimentary canal varies along its length from the basic pH value of the mouth, the high acidity of the stomach, and the neutral to slightly basic pH of the intestine (ca. 7.5). The capsules of the invention can be designed to disintegrate in a particular portion of the alimentary canal to deliver the encapsulated substance at that point.

In still another embodiment, the core particles contained in the capsules of the invention can subsequently be removed by dissolution in a medium in which the capsule wall is insoluble. The hollow capsules of the invention are useful to further incorporate substances for subsequent controlled release applications, including, but not limited to, biomaterials, such as cells and genetic material; bioactive agents and pharmaceuticals, such as small-molecule drugs, vaccines, antibodies, hormones, growth factors, sex sterilants, fertility inhibitors, fertility promoters, proteins, peptides, fragrances, flavors, vitamins, and nutrients; and chemical agents, such as nucleosides, nucleotides, oligonucleosides, oligonucleotides, agricultural materials (e.g., fertilizers and pesticides), preservatives, catalysts, enzymes, polymers, colorants and dyes (e.g., fluorescent compounds), sensor molecules, drug-formulation excipients, surfactants and detergents, and chemicals used in environmental remediation.

The capsules of the invention are useful in many areas, particularly for controlled release of encapsulated active ingredients under well-defined conditions, for example, in the fields of, without limitation, biotechnology; medicine; pharmaceuticals, such as controlled drug delivery; foods; agriculture; perfumery; personal care; and cosmetics.

### 4. BRIEF DESCRIPTION OF THE FIGURES

These and other features, aspects, and advantages of the present invention will become better understood with regard to the following description, examples, appended claims, and accompanying figures where:

FIG. 1 is an STEM image of capsules of the invention composed of polyethylene oxide/polymethacrylic acid;

FIG. 2 depicts fluorescence images of 10-layer polyethylene oxide/polymethacrylic acid capsules of the invention (panel A) and poly-N-vinylpyrrolidone/polymethacrylic acid capsules of the invention (panel B) at pH=2.0;

FIG. 3 depicts fluorescence images of cross-linked, 10-layer poly-N-vinylpyrrolidone/polymethacrylic acid capsules of the invention at pH=2 (panel A) and after exposure for 2 hours to pH=10 (panel B); and

FIG. 4 depicts fluorescence images of cross-linked, 10-layer polyethylene oxide/polymethacrylic acid capsules of the invention at pH=2 (panel A) and after exposure for 2 hours to pH=7 (panel B).

FIG. 5 depicts a fluorescence microscopy image of (polyethyleneimine/polymethacrylic acid)(poly-N-vinylpyrrolidone/polymethacrylic acid)(polyethyleneoxide/polymethacrylic acid)₃ capsules stained with Alexa Fluor 488 dihydrazide sodium salt fluorescent dye.

FIG. 6 depicts a fluorescence microscopy image of (polyethyleneimine/polymethacrylic acid)(poly-N-vinylpyrrolidone/polymethacrylic acid)₄ capsules stained with Alexa Fluor 488 dihydrazide sodium salt fluorescent dye.

FIG. 7 depicts a fluorescence microscopy image of (poly-N-vinylpyrrolidone/polymethacrylic acid)₄ capsules stained with Alexa Fluor 488 dihydrazide sodium salt fluorescent dye.

FIG. 8 depicts a fluorescence microscopy image of (polyethyleneimine/polymethacrylic acid)(poly-N-vinylpyrrolidone/polymethacrylic acid)(poly(N-isopropylacrylamide)/polymethacrylic acid)₂ capsules stained with Alexa Fluor 488 dihydrazide sodium salt fluorescent dye.

FIG. 9 depicts a fluorescence microscopy image of (polyethyleneimine/polymethacrylic acid)(polyvinylmethyl ether/polymethacrylic acid)₃ capsules stained with Alexa Fluor 488 dihydrazide sodium salt fluorescent dye. The initial template was cadmium carbonate.

FIG. 10 depicts a fluorescence microscopy image of (polyethyleneimine/polymethacrylic acid) (polyvinyl caprolactam/polymethacrylic acid)₃ capsules stained with Alexa Fluor 488 dihydrazide sodium salt fluorescent dye.

FIG. 11 schematically depicts covalent cross-linking of hydrogen-bonded multilayers via the carboxylic groups of polymethacrylic acid and the functional groups of a difunctional cross-linking reagent.

FIG. 12 schematically depicts covalent cross-linking of hydrogen-bonded multilayers.

### 5. DETAILED DESCRIPTION

In one embodiment, the capsules of the invention comprise layers of neutral polymeric films associated by hydrogen bonding on a core particle. In another embodiment, the capsules of the invention are hollow shells comprised of layers of neutral polymeric films associated by hydrogen bonding. In another embodiment of the invention, the hollow shells encapsulate a substance.

The shape of the capsules depends on variables, such as the shape of the core particles used in capsule formation and the mechanical and chemical properties of the capsule walls. The preferred average total volume of capsules of the invention is of from about 50 nm³ to about 50 mm³, more preferably, of from about 13,000 nm³ to about 60,000 μm³ even more preferably, of from about 60,000 nm³ to about 4,000 μm³, still even more preferably, 500,000 nm³ to about 1,000 μm³. Total volume means the entire volume of the capsule including the capsule walls.

Preferably, the capsules of the invention are substantially spherical. The preferred average diameter of capsules of the invention is of from about 3.5 nm to about 3.5 mm, more preferably, of from about 16 nm to about 1 mm, still more preferably, of from about 25 nm to about 40 μm, even more preferably, of from about 40 nm to about 20 μm, still even more preferably, 80 nm to about 10 μm. The diameter of the capsules means the entire diameter of the capsule including the capsule walls.

The thickness of the capsule shell (i.e. the polymer film layers), preferably, is of from about 5 nm to about 500 nm, more preferably, of from about 10 nm to about 30 nm.

The size and volume distribution of capsules of the invention depends to a large extent on the size and volume distribution of the core particles used in their formation. The size and volume distribution is readily controlled by one of skill in the art by selecting core particles with a certain size and volume distribution.

The capsules of the invention are useful to deliver the core particle or an encapsulated substance in a controlled and well-defined manner upon exposure to a particular external stimuli, such as a change in pH, salt concentration, temperature, solvent composition, application of an electric field, exposure to sunlight, or other external environmental change, depending on the specific composition of the capsules.

5.1 Multilayer Film Formation of the Capsule Shell on the Core Particle

The capsules of the invention containing a core particle can be prepared by adapting the layer-by-layer technique previously reported. See e.g., S. A. Sukhishvili et al, Layered, Erasable Polymer Multilayers Formed by Hydrogen-Bonded Sequential Self-Assembly, 35 MACROMOLECULES 301 (2002); G. B. Sukhorukov, et al., Layer-by-Layer Self Assembly of Polyelectrolytes on Colloidal Particles, 137 COLLOIDS SURF. A 253 (1998); G. D. Sukhorukov, et al., Stepwise Polyelectrolyte Assembly on Particle Surfaces: a Novel Approach to Colloid Design, 9 POLYM. ADV. TECHNOL. 759 (1998); F. Caruso, et al., Electostatic Self-Assembly of Silica Nanoparticle-Polyelectrolyte Multilayers on Polystyrene Latex Particles, 120 J. AM. CHEM. SOC. 8523 (1998); A. A Antipov, et al., Sustained Release Properties of Polyelectrolyte Multilayer Capsule, 105 PHYS. CHEM B 2281 (2001), all of which citations are hereby incorporated herein by reference.

In general, the capsules of the invention are prepared as follows. First, a solution of a first uncharged polymer to be adsorbed is contacted with the core particle's surface, and bonding of the polymer with the core particle's surface forms a first polymer layer. Next, a solution of the second polymer, of different identity than the first polymer, is contacted with the first layer, forming hydrogen bonds between the polymer layers, and forming a layered film. The process of contacting the surface with a solution of the first polymer and then a solution of the second polymer can be repeated until a film, of the desired thickness and number of layers, is formed around the core particle thereby forming a capsule of the invention. The surface of the growing capsules can be rinsed between applications to remove excess or non-bonded polymer. If more than two polymers are used to form the capsule, a solution of the additional polymers can be contacted with the growing film at any point of the process.

Aerosol deposition of polymer layers can also be used to prepare capsules of the invention. In another embodiment, multilayers of the invention can be prepared by sequential spraying of polymer solutions by adapting the procedures of J. B. Schlenoff. See e.g., J. B. Schlenoff et al., Sprayed Polyelectrolyte Multilayers, 16, LANGMUIR 9968 (2000), hereby incorporated herein by reference.

In still one more embodiment, the multilayer films of the invention can be formed by evaporating the first polymer and condensing it onto the core particles surface, followed by evaporation and condensation of the second polymer onto the surface, and repeating these steps until the desired thickness and layer number is achieved. Preferably, for such evaporation/condensation deposition, the polymers have a molecular weight of less than about 5000 g/mol, preferably, less than about 2000 g/mol.

For solution-phase deposition, the polymer concentration in the solvent is generally in the range of from about 0.01 mg/ml to about 0.5 mg/ml. Solvents for solution-phase deposition include any liquid in which the polymer is measurably soluble. Preferably, the solvent is an aqueous solution of appropriate pH. Other solvents useful in the invention include, but are not limited to, hydrocarbons such as pentane, hexane and toluene; alcohols such as methanol, ethanol, isopropanol, butanol, pentanol, and phenol; esters such butyl acetate; aldehydes and ketones such as formaldehyde, acetone and methyl ethyl ketone; dimethyl sulfoxide; carbonates such as propylene carbonate; amides and ureas, such as N-methyl formamide, tetramethylurea, dimethylacetamide, N-methylpyrrolidone, hexamethylphosphoric triamide and dimethyl formamide; supercritical fluids such as supercritical carbon dioxide; and mixtures thereof.

When an aqueous system is used for deposition, the appropriate pH of the deposition solution depends on the particular polymer system used. In general, the deposition pH is of from about 1 to about 5. One of skill in the art can readily select the pH appropriate for deposition based on two considerations: (a) the adhesion between the hydrogen-bonded polymer pair; and (2) insolubility of the core particle. The solution pHs, during the deposition and washing steps, can be controlled using a buffer, such as a phosphate buffer, of appropriate concentration, typically, a concentration of from about 0.005 M to about 0.1 M. The buffer's pH can be adjusted with an acid, such as hydrochloric acid, to produce buffers of the desired pH.

Optionally, to increase film adhesion of the first polymer layer, the core particles are pretreated using a primer layer. One of skill in the art will readily know the appropriate primer and procedure depending on the core particles and the chemical system (such as the surface charge and chemical nature of surface groups). In but one example, core particles with less propensity for forming hydrogen bonds can be treated with a solution of the first polymer in an aqueous medium at a concentration of from about 0.05 mg/ml to about 0.5 mg/ml at a pH of about 5 to about 7, depending on the polymeric system, followed by washing with buffer at pH=3.5.

Preferably, after every polymer deposition cycle, excess polymer is removed by: (a) centrifugation of the particle dispersion; (b) re-dispersing particles into a polymer-free solvent, preferably, the deposition solvent; and (c) repeating this washing procedure at least twice. The formation of multilayer capsules can be followed by fluorescence optical microscopy, as well known in the art. See e.g., G. B. Sukhorukov, et al., Microencapsulation By Means of Step-Wise Adsorption of Polyelectrolytes, 17 J. MICROENCAPSULATION 177 (2000), hereby incorporated herein by reference.

5.1.1 Determination of the Amounts of Polymers Deposited on Capsule Walls

Determination of the amounts of polymers deposited on the capsule walls can be accomplished using in-situ ATR-FTIR. The multilayer growth can be followed in a model system where polymers are deposited onto an appropriate flat surface. See e.g., S. A. Sukhishvili & S. Granick, Layered Erasable Polymer Multilayers Formed by Hydrogen-Bonded Sequential Self-Assembly, 35 MACROMOLECULES 301-310 (2002), hereby incorporated herein by reference.

Determination of the amounts of polymers deposited on capsule walls, can also be accomplished using Electron Energy Loss Spectrometry (EELS) to determine the thickness of capsule walls after the core dissolution using well-known techniques. See e.g., R. F. EGERTON, ELECTRON ENERGY-LOSS SPECTROSCOPY IN THE ELECTRON MICROSCOPE (2nd ed., 1996); V. Kozlovskaya, et al., Hydrogen-Bonded Polymer Capsules Formed by Layer-by-Layer Self-Assembly, 36 MACROMOLECULES 8590-8592 (2003), each of which citations is hereby incorporated herein by reference.

5.2 Core Particles of the Invention

In general, any surface can be coated with the hydrogen-bonded multilayer neutral polymeric films according to the methods of the invention to form capsules of the invention. Preferably, the surface is particulate material, more preferably, micro or nano-sized particulate material (“core particles”). One of skill in the art, can readily determine appropriate conditions for multilayer film deposition depending on the particles' identity and the polymer system. If the capsules of the invention are formed by solution-phase, layer-by-layer deposition, preferably, the core particles are substantially insoluble under the deposition conditions.

The size and shape of the core particles will vary depending on the size and shape of the capsules desired, the application in which the capsules of the invention will be used, the number of layers to be added to the core particles, and the chemical and physical properties of the polymer system.

The preferred average total volume of the core particles is of from about 50 nm³ to about 50 mm³, more preferably, of from about 4000 nm³ to about 1 mm³, still more preferably, of from about 13,000 nm³ to about 64,000 μm³, even more preferably, of from about 60,000 nm³ to about 8,000 μm³, still even more preferably, 500,000 nm³ to about 1000 μm³.

The preferred average diameter of the core particles is of from about 3.5 nm to about 3.5 mm, more preferably, of from about 16 nm to about 1 mm, still more preferably, of from about 25 nm to about 40 μm, even more preferably, of from about 40 nm to about 20 μm, still even more preferably, 80 nm to about 10 μm.

Core particles useful in the invention include, but are not limited to, crystalline materials, amorphous materials, lyophilized materials, spray-dried materials, and/or milled materials including, but not limited to, minerals, inorganic salts, small-molecule organic compounds, and organic macromolecules. Suitable core particles include pharmaceuticals, perfumes, cells, flavors, dyes, vitamins, nutrients, hormones, growth factors, and preservatives.

Suitable core particles further include porous materials, such as salts and minerals. See e.g., A. A. Antipov et al., Carbonate Microparticles for Hollow Polyelectrolyte Capsules Fabrication, 224 COLLOIDS SURF. A 175 (2003), hereby incorporated herein by reference. According to one aspect of the invention, such porous core particles can incorporate within their porous structure substances including, but not limited to, pharmaceuticals, perfumes, cells, flavors, dyes, vitamins, nutrients, hormones, growth factors, and preservatives.

5.3 Covalent Cross-Linking

A greater degree of stability can be imparted to hydrogen-bonded multilayer capsules by introducing covalent cross-links between the multilayer walls, for example, cross-linking based on known carbodiimide chemistry. Cross linking of capsules of the invention can be accomplished by adapting the methods of M. Adamczyk et al., Immunoassay Reagents for Thyroid Testing I. Synthesis of Thyroxine Conjugates 5 BIOCONJUGATE CHEM. 459 (1994); see also, T. Serizawa et al., Thermoresponsive Ultrathin Hydrogels Prepared by Sequential Chemical Reactions 35 MACROMOLECULES 2184 (2002), both of which citations are hereby incorporated herein by reference.

5.4 Polymers for Use in the Invention

Polymers for use in the invention include polymers containing hydrogen-bond donors and/or hydrogen-bond acceptors. Hydrogen-bond donors are moieties that contain at least one hydrogen atom that can participate in hydrogen-bond formation and a more electronegative atom bound to the hydrogen atom. Examples of these moieties include, but are not limited to, O—H, N—H, P—H, and S—H. The moiety C—H can also be a hydrogen-bond donor if the carbon atom is bound to another atom through a triple bond, if the carbon atom is bound through a double bond to O, or if the carbon atom is bound to at least two atoms selected from O, F, Cl, and Br.

Hydrogen-bond acceptors are moieties that contain an atom more electronegative than hydrogen that also contain a lone pair of electrons. Examples of such atoms include, but are not limited to, N, O, F, Cl, Br, I, S, and P. Examples of hydrogen-bond acceptor moieties include, but are not limited to, C═O, O—H, N—H, C—F, P═O, and C≡N.

Polymers having hydrogen-bond donors include, but are not limited to, polycarboxylic acids, such as polyacrylic acid and polymethacrylic acid; polynucleotides, such as poly(adenylic acid), poly(uridylic acid), poly(cytidylic acid), poly(uridylic acid), and poly(inosinic acid); polymers of vinyl nucleic acids, such as poly(vinyladenine); polyamino acids, such as polyglutamic acid and poly(E-N-carbobenzoxy-L-lysine); and polyalcohols, such as poly(vinyl alcohol); and copolymers thereof.

Examples of hydrogen-bond acceptors include, but are not limited to, polyethers such as polyethylene oxide, poly(1,2-dimethoxyethylene), poly(vinylmethyl ether), and poly(vinylbenzo-18-crown-6); polyketones and polyaldehydes, such as polyvinyl butyral and poly(N-vinyl-2-pyrrolidone); polyacrylamides, such as polyacrylamide, polymethacrylamide, and poly(N-isopropylacrylamide); polyamines, such as poly(4-amine)styrene; polyesters such poly(cylohexane-1,4-dimethylene terephthalate) and polyhydroxy methyl acrylate; polyphosphazenes, such as poly(bis(methylamino)phosphazene) and poly(bis(methoxyethoxyethoxy)phosphazene; and polysaccharides such as carboxymethyl cellulose; and copolymers thereof.

Preferably, the polymers of the invention comprise charge-forming structures, which are moieties that can develop charge when exposed to one or more environmental changes. Examples of environmental changes are a change in pH, a change in ionic strength, exposure to an electric field, or exposure to dissolved ions. Examples of moieties that can develop charge under changing pH conditions include acid or base moieties. Examples of moieties that can develop charge under exposure to an electric field include carboxylic acids. Examples of moieties that can develop charge under exposure to dissolved ions include crown ethers (upon exposure to certain alkali metal ions).

Examples of polymer systems for forming capsules of the invention include those of types 1 in the Table below:

5.4.1 Type 1: Homopolymer of Polycarboxylic Acid, Paired with the Specified Polymer B

| Polymer A | Polymer B |
|---|---|
| Polycarboxylic acid | Polyethylene oxide |
| Polycarboxylic acid | Poly(1,2-dimethoxyethylene) |
| Polycarboxylic acid | Poly(vinylmethyl ether) |

In the above three examples of Polymer B, the motif of proton acceptance in hydrogen bonding, is O . . . HO.

| Polymer A | Polymer B |
|---|---|
| Polycarboxylic acid | Poly(N-vinyl-2-pyrrolidone) (PVP) |
| Polycarboxylic acid | Poly(vinyl alcohol) |
| Polycarboxylic acid | Polyacrylamide |
| Polycarboxylic acid | Poly(-N-isopropylacrylamide) |
| Polycarboxylic acid | (CH₂(NCOCH₃)CH₂)x |

In the above five examples, summarized in the table, of Polymer B, the motif of proton acceptance, is NC═O . . . HO.

Capsules of the invention formed from such polymers systems can be dissolved at high pH or by dissolution. A film formed from polymethacrylic acid-PVP is stable in tetramethylurea and dimethylformamide and dissolves in dimethylacetamide, N-methylpyrrolidone, or hexamethylphosphoric triamide. The stability of these films can be affected by temperature; these films become more stable as the temperature increases in water, but are destabilized as the temperature increases in DMF.

5.4.2 Type 2: Multilayers that Include Crown Ethers as one Constituent

| Polymer A | Polymer B |
|---|---|
| Polycarboxylic acid | Vinyl polymers containing crown ether groups |
| | (for example: Poly(vinylbenzo-18-crown-6)) |

This type of film is destroyed by either high or low pH, depending on the specific monovalent ion present in the environment. For example, there is strong sensitivity to the type of cation complexed by the crown ether.

| Solution pH | Cation Present | Stability of Film |
|---|---|---|
| low pH | Li (for example LiCl) | stable |
| low pH | Na (for example NaCl) | intermediate |
| low pH | K, Cs or Ba (for example KCl, CsCl, | dissolves |
| | or BaCl₂) | |
| high pH | Li (for example LiOH) | dissolves |
| high pH | Na (for example NaOH) | dissolves |
| high pH | K or Cs (for example KOH or CsOH) | stable |

5.4.3 Type 3: Films that Include as One Constituent, Molecules Containing P═O Moieties. The Motif of Hydrogen Bonding is P═O . . . HO

| Polymer A | Polymer B |
|---|---|
| Polycarboxylic acid | —P═O containing polymers (for example |
| | polydimethyltetramethylene-phosphoric triamide) |

For example, the above film dissolves in hexamethylphosphoric triamide.

5.4.4. Type 4: Multilayers that Include Amino Acids as One Constituent

| Polymer A | Polymer B |
|---|---|
| Polyglutamic acid | Poly(vinyl alcohol) |
| Polyglutamic acid | Polyethylene oxide |
| Poly(E-N-carbobenzoxy-L-lysine) | Polyethylene oxide |

5.4.5 Type 5: Films Based on Hydrogen Bonding Between Synthetic Polynucleotides or Vinyl-Type Polymers Containing Nucleic Acid Bases

| Polymer A | Polymer B |
|---|---|
| Poly(adenylic acid) | Poly(uridylic acid) |
| Poly(cytidylic acid) | Poly(inosinic acid) |
| Poly(vinyladenine) | Poly(uridylic acid) |

These nucleic acid polymers will also form films with naturally occurring RNA (ribonucleic acid), DNA (deoxynucleic acid), as well as synthetic polynucleotides.

5.5 Hollow Capsules of the Invention

In certain instances, as discussed above, it is desirable to remove, e.g., by dissolution, the core particle from the capsules of the invention to give hollow capsules of the invention. The core of the polymer-covered particles is dissolved away by exposing the particles to a solution wherein the core particle is partially or substantially soluble, but in which the capsule walls are substantially insoluble. The particles are treated for a time sufficient to substantially remove the core, generally, for about 30 minutes to 60 minutes.

Suitable core particles useful for practice of this embodiment of the invention, include, but are not limited to, inorganic compounds such as calcium carbonate, cadmium carbonate, manganese carbonate; and organic compounds such as melamine formaldehyde, polysterene sulfonate latex particles, dyes, or pharmaceuticals.

The preferred average total volume of the cavity is of from about 50 nm³ to about 50 mm³, more preferably, of from about 4000 nm³ to about 1 mm³, still more preferably, of from about 13,000 nm³ to about 64,000 μm³, even more preferably, of from about 60,000 nm³ to about 8,000 μm³, still even more preferably, 500,000 nm³ to about 1000 μm³.

The preferred average diameter of the cavity is of from about 3.5 nm to about 3.5 mm, more preferably, of from about 16 nm to about 1 mm, still more preferably, of from about 25 nm to about 40 μm, even more preferably, of from about 40 nm to about 20 μm, still even more preferably, 80 nm to about 10 μm.

In another aspect of this embodiment, such soluble core particles comprise two or more substances, one or more of which substances can be removed in a subsequent step by the above-described dissolution while the other substance remains in the capsule. Suitable core particles for use in this embodiment of the invention include, but are not limited to, porous inorganic particles (such as porous calcium carbonate or porous magnesium carbonate) incorporating bioactive materials, such as pharmaceuticals, perfumes, cells, flavors, dyes, vitamins, nutrients, hormones, growth factors, and preservatives. See e.g., A. A. Antipov et al., Carbonate Microparticles for Hollow Polyelectrolyte Capsules Fabrication, 224 COLLOIDS SURF. A 175 (2003), hereby incorporated herein by reference.

5.6 Encapsulation of Substances into Capsules of the Invention

The capsule walls of capsules of the invention can be tailored such that upon exposure to a particular external stimulus, such as a change in pH, salt concentration, temperature, solvent composition, application of an electric field, or other external environmental change, they can encapsulate a substance. For example, the capsules of the invention can encapsulate a substance by becoming reversibly permeable (“open state”) to allow penetration by the substance to be encapsulated. The permeability can then be reversed (“closed state”), thereby encapsulating the substance. This is accomplished by exposing the capsules of the invention to the appropriate conditions depending on the capsule system. See e.g., G. B. Sukhorukov et al., pH-Contolled Macromolecule Encapsulation in and Release from Polyelectrolyte Multilayer Nanocapsules, 22 MACROMOL. RAPID COMMUN. 44 (2001); Antipov et al., Polyelectrolyte Multilayer Capsule Permeability Control, 200 COLLOIDS AND SURFACES A: PHYSIOCHEM. ENG. ASPECTS 198 (2002); WO 02/17888 (published Mar. 7, 2002), each of which citations is hereby incorporated herein by reference.

In but one example, capsules of the invention formed of poly-N-vinylpyrrolidone/polymethacrylic acid can be exposed to fluorescein isothiocyanate dextran 70,000-conjugate solution (concentration about 1 mg/ml) at a pH of about 6.0 for about 20 minutes followed by exposure of the capsules to a buffer of pH 2.0 for 5 minutes to encapsulate the dextran conjugate. The solution pHs, during exposure can be controlled using 0.01 M phosphate buffer. Relevant permeability data for this system is set forth in Table below.

TABLE
Permeability Data For Encapsulate/Release Of Dextran Conjugate

| | | | pH at which | pH at which |
|---|---|---|---|---|
| | | Encapsulation | capsules are | capsules are |
| Polymer A | Polymer B | substance | permeable | impermeable |
| poly-N- | polymethacrylic acid | Fluorescein | 6.0 | 2.0 |
| vinylpyrrolidone | | isothiocyanate Dextran | | |
| | | 70,000-Conjugate | | |
| polyethylene oxide | polymethacrylic acid | Fluorescein | 4.4 | 2.0 |
| | | isothiocyanate Dextran | | |
| | | 70,000-Conjugate | | |

5.6.1 Encapsulation Substances

Any substances can be introduced into capsules of the invention, for example, into the hollow capsules of the invention prepared according to Section 5.5, using the above-described procedures and/or other known literature procedures. Substances useful to incorporate into capsules of the invention include, but not limited to, biomaterials, such as cells and genetic material; bioactive agents and pharmaceuticals, such as small-molecule drugs, vaccines, antibodies, hormones, growth factors, sex sterilants, fertility inhibitors, fertility promoters, proteins, peptides, fragrances, flavors, vitamins, and nutrients; and chemical agents, such as nucleosides, nucleotides, oligonucleosides, oligonucleotides, agricultural materials (e.g., fertilizers and pesticides), preservatives, catalysts, enzymes, polymers, colorants and dyes (e.g., fluorescent compounds), sensor molecules, drug-formulation excipients, surfactants and detergents, and chemicals used in environmental remediation.

Substances suitable for incorporation and/or encapsulation, for subsequent controlled release under the appropriate conditions include, but are not limited to, oligomeric and polymeric molecules, such as natural and synthetic polypeptides, oligo- and polynucleotides or synthetic water-soluble polymers, such as heparin, insulin, calcitonin, cromolyn, human growth factors, and hormones; polycations; basic growth factors, such as fibrinoblast growth factor-2 (FGF2), insulin-like growth factor IGF-I, spermine and chitosane; synthetic polycarboxylic acids, such as poly(styrenesulfonic acid) and poly(phosporic acid); proteins such as albumins and main soy protein; heparin-binding proteins; growth factors, such as fibrinoblast growth factor-1 (FGF1) and insulin-like growth factor IGF-II; tissue-type plasminogen activators (t-PA), such as monteplase; cofactors such as heparin cofactor II hyaluronic acid, heparin and DNA and RNA molecules; antibiotics such as, pivampicillin and cephaloridine; antiinflammatory agents, such as glaphenine aspirin, fenamic acids (flufenamic and mefanamic acids), ibuprofen, flibuprofen, naproxen and indomethacin; anesthetics, such as ecgoninic acid, benzocaine, procaine and piridocaine; hormones; neutrotransmitters; humoral factor, such as amphetamine prostoglandines (dinoprost, PGE₁, PGF_{1α}, PGF_{2α} and PGE₂) and meparfynol; antidepressants and tranquilizers, such as dibenzoxepins, etryptamine, methpimazine, and pipamazine; antispasmodic agents, such as methantheline bromide, propanetheline bromide and fenethylline; miscellaneous pharmaceuticals, such as hycanthone; antihypertensive agents, such as bretylium tosylate, dihydralazine and bretylium tosylate; anesthetics and central nervous system stimulants, such as neostigmine, ephedrine, oxyfedrine, levonordefrine, amphetamine, tranylcypromine, fencamfine, and hydroxyamphetamine; antidepressants, such as phenelzine and pheniprazine; antidiabetic agents, such as phenformin; antibiotics, such as acephylline, carbencillin, cephalothin, nafcillin, methicillin and penicillin G ethionamide, protonsil, sulfanilamide, and sulfanilamide derivatives; antiinfective agents, such as chlorazanil, aminophenazole, trimethoprim, pyrimethamine, primaquine, and sontoquine; analgetics, such as phenazopyridine; hypotensive agents, such as minoxidil; obesity-control agents, such as phentermine and chlorphentermine; diuretic agents, such as ethacrynic acid, probenecid chlorazanil, aminotetradine, amiloride, and amisotetradine; anticoccidial pharmaceuticals, such as amprolium; anthelmentic agents, such as dithiazinine; neurotoxins; vitamins, such as thiamine (B₁), nicotinamide (B₃), pyridoxamine (B₆), and pantothenic acid (B₅); estrogens (methallenestril); enzyme inhibitors, such as nodularin and its synthetic derivatives cyclo[-(3S,E)-3-phenylethenyl-3-aminopropanoyl-α-(R)-Glu-α-OH-γ-Sar-(R)-Asp-α-OH-β-(S)-Phe-] and cyclo[-(2S,3S,E)-2-methyl-3-phenylethenyl-3-aminopropanoyl-β-(R)-Glu-α-OH-γ-Sar-(R)-Asp-α-OH-β-(S)-Phe-]; muscle relaxants, such as phenyramidol; and cofactors, such as biotin and trombomodulin.

5.7 Release of Core Particles or Encapsulated Substances

The capsule walls can be eroded or made permeable in order to expose the core particles or the encapsulated substances to the surrounding environment so that the substance is released into the surrounding environment. For example, the core particles or other encapsulated material can be released from the capsules of the invention by exposing them to an external stimuli such as a change in pH, salt concentration, temperature, solvent composition, application of an electric field, exposure to sunlight, or other external environmental change, depending on the specific composition of the capsules. For example, pH-triggered capsule decomposition or pH-induced permeability changes can be used for oral drug-delivery or for delivery through the mucous membrane, for example, delivery of antibacterial agents for treatment of vaginal infections. Temperature triggering of capsules of the invention can be used for transdermal or intradermal drug delivery.

The procedures described in the literature can be adapted for release of the core particles or substances encapsulated in the capsules of the invention. See e.g., S. A. Sukhishvili et al., Layered, Erasable, Ultrathin Polymer Films, 122 J. AM. CHEM. SOC. 955 (2000); Shchukin et al., Micron-Scale Hollow Polyelectrolyte Capsules with Nanosized Magnetic Fe₃O₄ Inside, 57 MATERIALS LETTERS 1743 (2003), each of which citations is hereby incorporated herein by reference. Alternatively, depending on the polymer system, heating or the addition of solvents can induce release of an encapsulated substance, for example, using the procedure described in Shi et al., Release Behavior of Thin-Walled Microcapsules Composed of Polyelectrolyte Multilayers, 17 LANGMUIR 2036 (2001), hereby incorporated herein by reference.

Preferably, release of the encapsulated substance can be accomplished by subjecting the capsules to a pH environment at which the capsule wall releases the substance.

The critical pHs of some exemplary polymer systems of the invention are provided in the Table below.

TABLE
Critical pH of Capsules Invention

| | Approximate Critical |
|---|---|
| Polymer System | pH |
| poly-N-vinylpyrrolidone/polymethacrylic acid | 6.9 |
| polyethylene oxide/polymethacrylic acid | 4.6 |
| poly-N-acrylamide/polymethacrylic acid | 5.0 |
| poly-N-isopropylacrylamide/polymethacrylic acid | 5.5 |
| poly-N-vinylcaprolactam/polymethacrylic acid | 6.9 |
| poly-N-vinylpyrrolidone/poly(acrylic) acid | 5.9 |
| polyethylene oxide/poly(acrylic) acid | 3.6 |
| poly-N-acrylamide/poly(acrylic) acid | 4.0 |
| poly-N-isopropylacrylamide/poly(acrylic) acid | 4.5 |
| poly-N-vinylcaprolactam/poly(acrylic) acid | 5.9 |

Dissolution of the capsule walls is accomplished by placing them in an environment at the critical pH or higher.

5.8 Additives Incorporated Within and Between the Walls of the Capsules of the Invention

Any agent can be incorporated within and between the walls of the capsules of the invention. The additives can be incorporated by known literature methods, for example, Nicol et al., Polyelectrolyte Multilayers as Nanocontainers for Functional Hydrophilic Molecules, 19 LANGMUIR 6178 (2003), hereby incorporated herein by reference. Preferably, for layer-by-layer solution-phase deposition, the substance to be incorporated is dissolved or dispersed in the deposition solvent in which a polymer of the invention is dissolved. Alternatively, the substance can be incorporated in the capsule wall if the substance can be evaporated and condensed with one of the polymers.

5.8.1 Bioactive Agents and Pharmaceuticals as Additives

The bioactive agents can be any physiologically or pharmacologically active substance or substances optionally in combination with pharmaceutically acceptable carriers and additional ingredients such as antioxidants, stabilizing agents, permeation enhancers, etc. The bioactive agents can be any of the agents that are known to be delivered to the body of a human, animal, insect, or plants. Preferably, the bioactive agents used in the invention are soluble in water. Suitable bioactive agents include, but are not limited to, biomaterials, such as cells; bioactive agents and pharmaceuticals, such as small-molecule drugs, vaccines, antibodies, hormones, growth factors, proteins, peptides, and genetic material; vitamins; nutrients; agricultural materials, such as fertilizers and pesticides; fragrances; flavors; preservatives; catalysts, such as enzymes; and polymers; sex sterilants, fertility inhibitors, and fertility promoters.

Specific examples of bioactive agents for use in the invention include, but are not limited to, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, and cephalexin hydrochloride.

### 5.9 EXAMPLES

### 5.9.1 Example 1

### Preparation of Capsules of the Invention: Poly-N-Vinylpyrrolidone, Polymethacrylic Acid and Polyethylene Oxide

This example details the preparation of: (1) capsules comprising alternating layers of poly-N-vinylpyrrolidone (Mw 55,000) and polyethylene oxide (Mw 200,000); and (2) capsules comprising alternating layers of polymethacrylic acid (Mw 150,000) and polyethylene oxide (Mw 200,000).

The core particle was cadmium carbonate particles (CdCO₃), which was synthesized by mixing equal amounts of 1 M cadmium nitrate solution and 2 M urea solution followed by heating the mixture for 16 hours at 90° C. The resulting crystals were rhombohedral and ranged in size from 0.1 to 10 μm.

The poly-N-vinylpyrrolidone/polymethacrylic acid or polyethylene oxide/polymethacrylic acid multilayers were then prepared using the layer-by-layer technique with a centrifugation set-up as described in G. B. Sukhorukov, et al., Layer-by-Layer self assembly of polyelectrolytes on colloidal particles, 137 COLLOIDS SURF. A 253 (1998); G. D. Sukhorukov, et al., Stepwise Polyelectrolyte Assembly on Particle Surfaces: a Novel Approach to Colloid Design, 9 POLYM. ADV. TECHNOL. 759 (1998); F. Caruso, et al., Electostatic Self-Assembly of Silica Nanoparticle-Polyelectrolyte Multilayers on Polystyrene Latex Particles, 120 J. AM. CHEM. SOC. 8523 (1998); A. A Antipov, et al., Sustained Release Properties of Polyelectrolyte Multilayer Capsule, 105 PHYS. CHEM B 2281 (2001), all of which citations are hereby incorporated herein by reference.

Polymers were deposited from 0.2 mg/ml solutions. The solution pHs, during the deposition and washing steps, were controlled using 0.01 M phosphate buffer whose pH was adjusted with hydrochloric acid to produce acidic solutions. To increase film adhesion, CdCO₃ particles were pretreated with a 0.2 mg/ml solution of polymethacrylic acid at pH=7.0, followed by washing with buffer at pH=3.5. The deposition of poly-N-vinylpyrrolidone/polymethacrylic acid or polyethylene oxide/polymethacrylic acid layers was then continued at pH=3.5, starting from the poly-N-vinylpyrrolidone or polyethylene oxide layer.

After every polymer deposition cycle, excess polymer was removed by: (a) centrifugation of the particle dispersion; (b) re-dispersing particles into a polymer-free buffer solution; and (c) repeating this washing procedure at least twice. Starting from the second layer, buffer at pH=3.5 was used at every washing step. In neither system was severe particle aggregation observed. Gently shaking the precipitate was usually sufficient to re-disperse the particles after centrifugation. On occasion, the precipitate was sonicated for one minute to reverse aggregation. In a typical experiment, ten polymer layers were deposited, with poly-N-vinylpyrrolidone or polyethylene oxide as the outermost layer.

The CdCO₃ core of the polymer-covered particles was then dissolved by exposing the particles to buffer solution at pH=1.1. The time allowed to completely remove the core was 30 minutes.

To determine the amounts of polymers deposited on capsule walls, two strategies were used. First, using in situ ATR-FTIR the multilayer growth was followed in a model system where polymers were deposited onto a flat surface of oxidized Si. The oxidation of the surface, priming with the first layer, multilayer deposition, as well as calculation of the amount adsorbed were done as described in S. A. Sukhishvili & S. Granick, Layered Erasable Polymer Multilayers Formed by Hydrogen-Bonded Sequential Self-Assembly, 35 MACROMOLECULES 301-310 (2002), hereby incorporated herein by reference. The result of these ATR-FTIR studies gave the total amounts adsorbed of 56 mg/m² and 36 mg/m² for 10-layer polyethylene oxide/polymethacrylic acid and poly-N-vinylpyrrolidone/polymethacrylic acid systems, respectively.

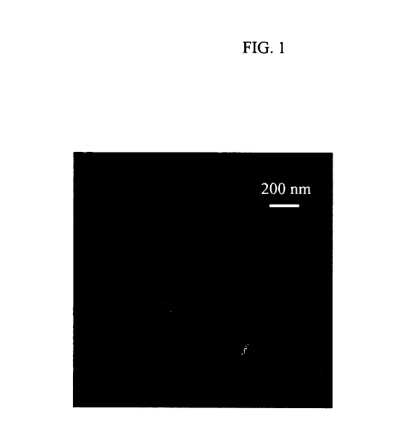

Second, Electron Energy Loss Spectrometry (EELS) was used to determine the thickness of capsule walls after the core dissolution. FIG. 1 shows a STEM image of polyethylene oxide/polymethacrylic acid capsules. More specifically, FIG. 1 is a high-angle annular-dark-field STEM image of polyethylene oxide/polymethacrylic acid capsules (bright contrast) on a lacy-carbon TEM support film. The dark areas represent pores in the support film.

The table below summarizes the average multilayer film thicknesses, determined from at least twelve different capsules per specimen.

| | Thickness |
|---|---|
| Polymer System | (nm) |
| polyethylene oxide/polymethacrylic | 16 ± 3 |
| acid | |
| poly-N- | 18 ± 4 |
| vinylpyrrolidone/polymethacrylic acid | |

PEELS spectra were collected at 20 nm intervals along line scans that transected individual multilayer capsules. The average capsule wall thickness was obtained by averaging ten measurements from a given capsule and at least 12 capsules in each of the two specimens (polyethylene oxide/polymethacrylic acid and poly-N-vinylpyrrolidone/polymethacrylic acid) were studied.

The capsules were deposited from buffer solution at pH=3.5 onto a lacy-carbon TEM support film, dried, cooled to −165° C. and analyzed using a 200 keV Philips CM20 field emission, scanning-transmission electron microscope (FEG-STEM) with a Gatan 776 Enfina PEELS spectrometer. The capsule-wall thickness was derived using the relation: 2t/λ=ln(It/I0), where t is thickness, λ is the mean free path (MFP) for total inelastic electron scattering of the polymer, It and I0 are the total and the zero-loss spectral intensities, respectively; we used λ=260 nm based on measurements of inelastic electron scattering in polystyrene. It can be seen from the Table above that a 10-layer thickness was significantly smaller when deposited onto a CdCO3 core, as compared to the model Si surface (16 nm versus 56 nm for polyethylene oxide/polymethacrylic acid system and 18 nm versus 36 nm for poly-N-vinylpyrrolidone/polymethacrylic acid system, assuming a density of 1 g/cm³).

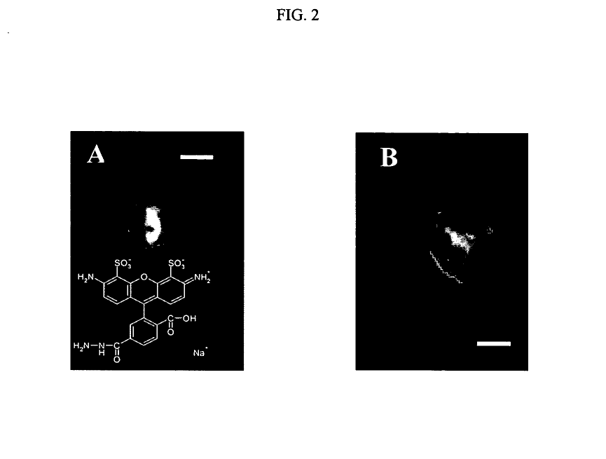

The formation of multilayer capsules was followed by fluorescence optical microscopy using a Nikon Eclipse E1000 microscope with 40×LU Plan objective lens. All images were stained with a high-quantum yield photostable Alexa 488 Fluor hydrazide fluorophore (commercially available, for example, from Molecular Probes, Inc. Eugene, Oreg.) The structural formula of this label is shown in FIG. 2. This label noncovalently attaches to the functional groups of the capsule wall over a wide interval of pH, due to electrostatic, hydrogen bonding as well as Van der Waals interactions, allowing fluorescent imaging of the hydrogen-bonding capsules. However, the staining is more efficient at acidic pH values, when the label is probably able to form hydrogen bonds with un-ionized carboxylic groups.

FIG. 2 shows fluorescence images of a 10-layer polyethylene oxide/polymethacrylic acid (panel A) and poly-N-vinylpyrrolidone/polymethacrylic acid capsules (panel B) at pH=2.0. The inset shows the chemical structure of Alexa Fluor 488 hydrazide fluorophore. A 0.02 M Alexa Fluor 488 hydrazide sodium salt solution has been used for staining capsules. The bar corresponds to 4 μm. It can be seen that robust capsules are produced.

### 5.9.2 Covalent Crosslinking of Poly-N-Vinylpyrrolidone/Polymethacrylic Acid and Polyethylene Oxide/Polymethacrylic Acid Capsules

The polymer walls of the capsules of the invention as prepared above were cross linked by adapting known carbodiimide chemistry. The carboxylic groups were activated with 5 mg/ml of 1-ethyl-3-(3-dimethylaminopropyl)-carbodiimide hydrochloride (EDC) solution at pH 5.0 (for poly-N-vinylpyrrolidone/polymethacrylic acid multilayers) or pH 4.0 (for polyethylene oxide/polymethacrylic acid multilayers), followed by reacting with 0.01 mg/ml of ethylenediamine at pH 5.8 or 4.0 for poly-N-vinylpyrrolidone/polymethacrylic acid or polyethylene oxide/polymethacrylic acid multilayers, respectively. The cadmium carbonate core was removed after cross-linking by exposing the modified particles to pH=1.1, leaving behind cross-linked capsule walls.

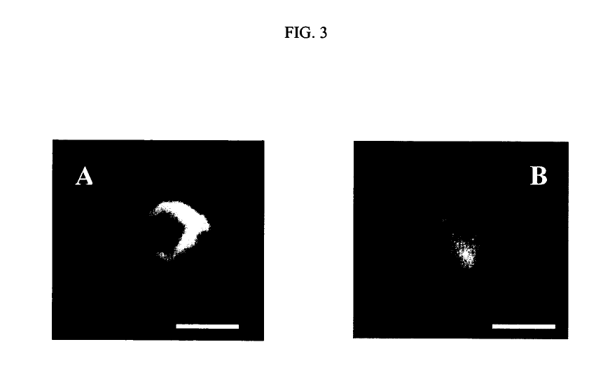

FIG. 3 is fluorescence images of cross-linked 10-layer poly-N-vinylpyrrolidone/polymethacrylic acid capsules at pH=2 (panel A) and after exposure for 2 hours to pH=10 (panel B). The bar corresponds to 4 μm. FIG. 3 shows that after the cross-linking treatment, poly-N-vinylpyrrolidone/polymethacrylic acid capsules became stable at pH=10. Cross-linked capsules stored for several months at pH=10 did not show any signs of disintegration.

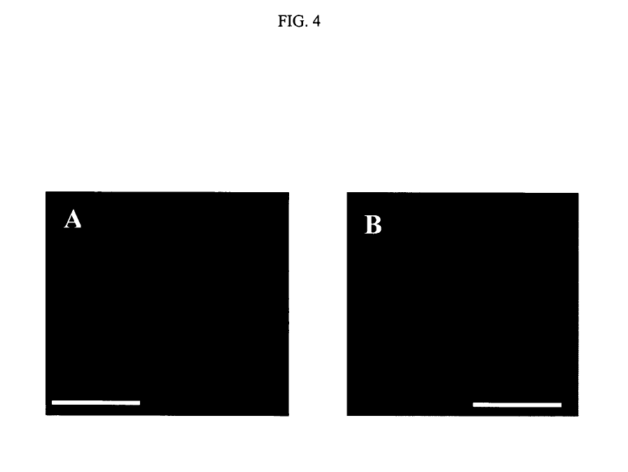

As shown in FIG. 4, the pH-stability of polyethylene oxide/polymethacrylic acid capsules was also greatly improved by cross linking. FIG. 4 shows fluorescence images of cross-linked 10-layer polyethylene oxide/polymethacrylic acid capsules at pH=2 (panel A) and after exposure for 2 hours to pH=7 (panel B). The bar corresponds to 4 μm. Capsules could be observed for at least six days when exposed to pH=7.

### 5.9.3 Preparation of Poly(Ethylene Oxide)/Poly(Methacrylic Acid) Capsules Using Negatively Charged Cadmium Carbonate or Manganese Carbonate Particles as the Core Using Precursor Layers

Precursor layers of polyethyleneimine (Mw 600,000) and polymethacrylic acid (Mw 150,000) from 0.2 mg/ml solution at pH=3 were deposited on negatively charged cadmium carbonate or manganese carbonate particles by centrifugation with three washing cycles with water at pH=3.5. The pH of each water polymer solution was adjusted to 3.5 using 0.01 M HCl. The negatively charged cadmium carbonate particles were synthesized as described in A. Janekovic, et al., Preparation of Monodispersed Colloidal Cadmium Compounds, 103 J. COLLOID INTERFACE SCI. 436 (1985). The average cadmium carbonate particle size was 10 microns. The negatively charged manganese carbonate particles were synthesized as described in A. Antipov, et al., Urease-Catalyzed Carbonate Precipitation inside the Restricted Volume of Polyelectrolyte Capsules, 24 MACROMOL. RAPID COMMUN. 274 (2003). The average manganese carbonate particle size was 2 microns.

Then one bilayer of poly-N-vinylpyrrolidone/polymethacrylic acid was self-assembled on the resulting intermediate capsule at pH=3.5, as described above, starting from poly-N-vinylpyrrolidone.

Next, polyethyleneoxide/polymethacrylic acid layers were self-assembled at pH=3.5 starting from polyethyleneoxide via centrifugation. Each deposition cycle was followed by washing out excess of the polymers with a buffer solution at pH=3.5.

Centrifugation of the suspensions at 1200 rpm for 1 min removed the supernatant. After the desired number of layers was deposited, the carbonate core was dissolved and removed by treating the capsules with a 0.1 M aqueous HCl solution.

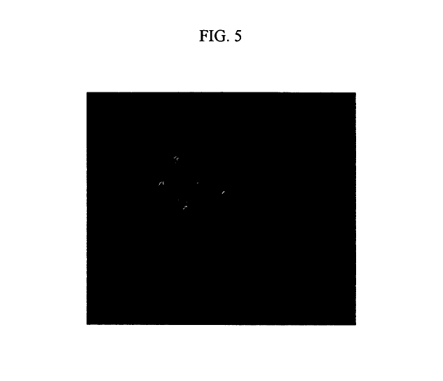

The resulting hollow capsules were washed several times with the HCl solution to wash away the products of the core dissolution. Specifically, the supernatant was removed after centrifugation at 2000 rpm for 10 minutes. The thickness of a single capsule wall of (polyethyleneimine/polymethacrylic acid)(poly-N-vinylpyrrolidone/polymethacrylic acid)(polyethyleneoxide/polymethacrylic acid)₄ composition was measured by Electron-Energy-Loss Spectrometry and made up 18±3 nm. The typical fluorescence microscopy image of the capsules of the kind is shown in FIG. 5, which is a fluorescence microscopy image of (polyethyleneimine/polymethacrylic acid)(poly-N-vinylpyrrolidone/polymethacrylic acid)(polyethyleneoxide/polymethacrylic acid)₃ capsules stained with Alexa Fluor 488 dihydrazide sodium salt fluorescent dye. The initial template was cadmium carbonate.

### 5.9.4 Preparation of Poly(Ethylene Oxide)/Poly(Methacrylic Acid) Capsules Using Positively Charged Cadmium Carbonate or Manganese Particles as the Core Using Precursor Layers

Precursor layers of polyethyleneimine (Mw 600,000) and polymethacrylic acid (Mw 150,000) from 0.2 mg/ml solution at pH=3.5 were deposited on negatively charged cadmium carbonate or manganese carbonate particles by centrifugation with three washing cycles with water at pH=3.5. The pH of each water polymer solution was adjusted to 3.5 using 0.01 M HCl. The positively charged cadmium carbonate particles were synthesized by mixing equal amounts of 1 M cadmium nitrate and 1 M sodium carbonate solutions. The average cadmium carbonate particle size was 1 micron. The positively charged manganese carbonate particles were synthesized as described in A. Antipov, et al., Urease-Catalyzed Carbonate Precipitation inside the Restricted Volume of Polyelectrolyte Capsules, 24 MACROMOL. RAPID COMMUN. 274 (2003). The average manganese carbonate particle size was 2 microns.

Then one bilayer of poly-N-vinylpyrrolidone/polymethacrylic acid was self-assembled on the resulting intermediate capsule at pH=3.5, as described above, starting from poly-N-vinylpyrrolidone.

Next, polyethyleneoxide/polymethacrylic acid layers were self-assembled at pH=3.5 starting from polyethyleneoxide via centrifugation. Each deposition cycle was followed by washing out excess of the polymers with a buffer solution at pH=3.5.

Centrifugation of the suspensions at 1200 rpm for 1 min removed the supernatant. After the desired number of layers was deposited, the carbonate core was dissolved and removed by treating the capsules with a 0.1 M aqueous HCl solution.

The resulting hollow capsules were washed several times with the HCl solution to wash away the products of the core dissolution. Specifically, the supernatant was removed after centrifugation at 2000 rpm for 10 minutes.

### 5.9.5 Preparation of Poly(Ethylene Oxide)/Poly(Methacrylic Acid) Capsules Using Positively Charged Cadmium Carbonate/Manganese Carbonate Particles as the Core Without Precursor Layers

First, the polymethacrylic acid was deposited on positively charged cadmium carbonate or manganese carbonate particles from 0.2 mg/ml buffer solution at pH=6.5 followed by triple washing with 0.01 M phosphate buffer at pH=3.5 as described above.

Next polyethyleneoxide/polymethacrylic acid layers were self-assembled at pH=3.5 starting from polyethyleneoxide as described above.

Each deposition cycle was followed by washing the excess polymer with a buffer solution at pH=3.5. Centrifugation of the suspensions at 1200 rpm for one min removed the supernatant.

After a desired number of layers were deposited, the carbonate core was dissolved to produce hollow capsules. Specifically, 0.1 M HCl solution was used to decompose the carbonate core.

The resulting hollow capsules were washed several times with the HCl solution to remove the core-decomposition products. Specifically, the supernatant was removed after centrifugation at 2000 rpm for ten minutes. The thickness of a single capsule wall of (polyethyleneoxide/polymethacrylic acid)₅ composition was measured by Electron-Energy-Loss Spectrometry and made up 16±3 nm.

5.9.6 Preparation of Polyethylene Oxide/Polymethacrylic Acid Capsules Using Silicon Dioxide Particles as the Core Without Precursor Layers

First, a polyethyleneoxide layer was deposited onto 4.0±0.2 μm SiO₂ particles (Polysciences Inc) from 0.2 mg/ml solution of polyethyleneoxide at pH=2.5 adjusted with 0.01 M HCl followed by triple washing with 0.01 M phosphate buffer at pH 2.5. Then a polymethacrylic acid layer was self-assembled at pH=2.5 as described above.

Each deposition cycle was followed by washing excess polymer with a buffer solution at pH=2.5. Centrifugation of the suspensions at 1000 rpm for 0.5 min was used to remove the supernatant.

After a desired number of layers were deposited, the silica core was dissolved to produce hollow capsules by exposing the covered particle suspension to 5% hydrofluoric acid/water solution for 3 hours. The resulting hollow capsules were washed tree times with the HF solution followed by several washings with buffer at pH=2 to wash away the remaining aqueous HF. The supernatants were removed after centrifugation at 2000 rpm for 10 minutes.

### 5.9.7 Preparation of Poly-N-Vinylpirrolidone/Polymethacrylic Acid via Positively or Negatively Charged Cadmium Carbonate or Manganese Carbonate Core Particles Using Precursor Layers

First, precursor layers of polyethyleneimine and polymethacrylic acid from 0.2 mg/mL solution at pH=3.5 were deposited as described above on either positively or negatively charged cadmium carbonate/manganese carbonate core particles (compositions described above) with three washing cycles at pH=3.5 in between.

Next, poly-N-vinylpyrrolidone/polymethacrylic acid layers were self-assembled at pH=3.5 starting from poly-N-vinylpyrrolidone as described above. Each deposition cycle was followed by washing out excess of the polymers with a buffer solution at pH=3.5. Centrifugation of the suspensions at 1200 rpm for 1 min was used to remove the supernatant. After a desired number of layers were deposited, the carbonate core was dissolved to produce hollow capsules. Specifically, 0.1 M HCl solution was used to decompose the particles.

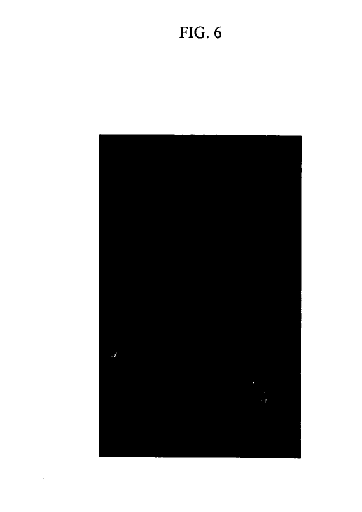

The resulting hollow capsules were washed several times with the 0.01 M HCl solution to wash away the products of the core decomposition products. The supernatant was removed after centrifugation at 2000 rpm for 10 minutes. The thickness of a single capsule wall of (polyethyleneimine/polymethacrylic acid)(poly-N-vinylpyrrolidone/polymethacrylic acid)₅ composition was measured by Electron-Energy-Loss Spectrometry and made up 44±5 nm. The typical fluorescence microscopy image of the capsules of the kind is shown in FIG. 6, which is a fluorescence microscopy image of (polyethyleneimine/polymethacrylic acid)(poly-N-vinylpyrrolidone/polymethacrylic acid)₄ capsules stained with Alexa Fluor 488 dihydrazide sodium salt fluorescent dye. The initial template was cadmium carbonate.

### 5.9.8 Preparation of Poly(N-Vinylpirrolidone)/Polymethacrylic Acid Poly-N-Vinylpyrrolidone/Polymethacrylic Acid Using Silicon Dioxide Particles as the Core Without Precursor Layers

First, a poly-N-vinylpyrrolidone layer was deposited onto 4 μm SiO₂ particles from 0.2 mg/mL solution of poly-N-vinylpyrrolidone at pH=1.6 followed by triple washing with buffer at pH=1.6, as described above.

Next, a polymethacrylic acid layer was self-assembled at pH=2. The self-assembly was performed as described above. Each deposition cycle was followed by washing excess polymer with a buffer solution at pH=1.6. Centrifugation of the suspensions at 1000 rpm for 0.5 min was used to remove the supernatant.

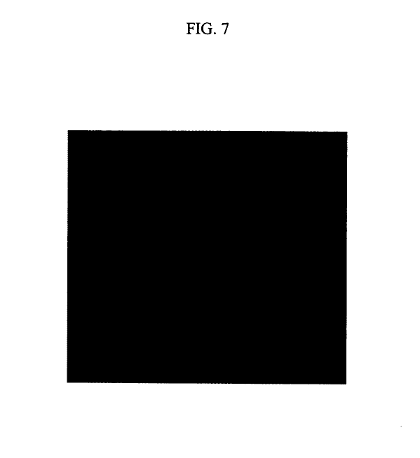

After a desired number of layers were deposited, the silica core was dissolved by exposing the covered particle suspension to 5% HF/water solution for 3 hours to decompose the particles and to produce the hollow capsules. The resulting hollow capsules were washed three times with the aqueous HF solution, followed by several washings with buffer at pH=2 to remove any remaining HF. During this washing, the supernatants were removed after centrifugation at 2000 rpm for 10 minutes. The typical fluorescence microscopy image of the capsules of the kind is shown in FIG. 7, which is a fluorescence microscopy image of (poly-N-vinylpyrrolidone/polymethacrylic acid)₄ capsules stained with Alexa Fluor 488 dihydrazide sodium salt fluorescent dye. The initial template was SiO₂.

The thickness of a single capsule wall of (poly-N-vinylpyrrolidone/polymethacrylic acid)₄ composition was measured by Electron-Energy-Loss Spectrometry and made up 31±4 nm.

### 5.9.9 Preparation of Temperature-Responsive Capsules of Poly-N-Isopropylacrylamide)/Polymethacrylic Acid

Precursor layers of polyethyleneimine and polymethacrylic acid from 0.2 mg/mL solution at pH=3.5 were deposited onto positively or negatively charged cadmium carbonate particles (16-20 μm) with three washing cycles at pH=3.5 as described above.

Next, one bilayer of poly-N-vinylpyrrolidone/polymethacrylic acid was self-assembled at pH=3.5 starting from poly-N-vinylpyrrolidone as described above. Then poly-N-isopropylacrylamide (Mw 300,000, Scientific Polymer Products, Inc.)/polymethacrylic acid layers were self-assembled at pH=3.5 starting from poly-N-isopropylacrylamide using the procedure described above. The deposition time was 15 minutes. Each deposition cycle was followed by removing excess polymer by washing with a buffer solution at pH=3.5. Centrifugation of the suspensions at 1200 rpm for 0.5 min removed the supernatant after each washing.

After the desired number of layers were deposited, the carbonate core was dissolved by treating the capsules with 0.1 M HCl solution as described above. The resulting hollow capsules were washed several times with the HCl solution to remove core-decomposition products. During the washings, the supernatant was removed after centrifugation at 2000 rpm for 10 minutes.

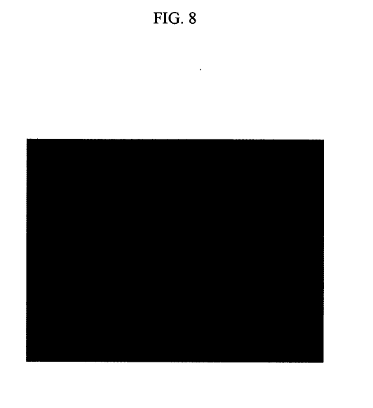

During the deposition cycles the polymer solutions were maintained at 5° C. by cooling in a refrigerator. Thickness of a single wall of the resulting (polyethyleneimine/polymethacrylic acid) (poly-N-vinylpyrrolidone/polymethacrylic acid) (poly-N-isopropylacrylamide/polymethacrylic acid)₂ capsules was determined by electron energy-loss spectrometry and made up 68.9±18.7 nm. The typical fluorescence microscopy image of the capsules of the kind is shown in FIG. 8, which is a fluorescence microscopy image of (polyethyleneimine/polymethacrylic acid) (poly-N-vinylpyrrolidone/polymethacrylic acid) (poly-N-isopropylacrylamide/polymethacrylic acid)₂ capsules stained with Alexa Fluor 488 dihydrazide sodium salt fluorescent dye. The initial template was cadmium carbonate.

### 5.9.10 Preparation of Temperature-Responsive Capsules of Polyvinylmethyl Ether/Polymethacrylic Acid

First, precursor layers of polyethyleneimine and polymethacrylic acid from 0.2 mg/ml solution at pH=3.5 were deposited onto positively or negatively charged cadmium carbonate particles (16-20 μm) with three washing cycles at pH=3.5 using the procedure described above.

Next, the pH of the suspension was adjusted to 4.5 by washing it with buffer at pH=4.5 three times. Then polyvinylmethyl ether/polymethacrylic acid layers were self-assembled at pH=4.5 starting from polyvinylmethyl ether using the procedure described above. Deposition time was 15 minutes. Each deposition cycle was followed by removing excess polymer by washing with a buffer solution at pH=4.5. Centrifugation of the suspensions at 1200 rpm for 0.5 min was used to remove the supernatant during each washing.

After a desired number of layers were deposited, the carbonate core was dissolved to produce hollow capsules as detailed above by way of a 0.1 M aqueous HCl solution. The resulting hollow capsules were washed several times with the aqueous HCl solution to remove core-decomposition products. During the washings, the supernatant was removed upon centrifugation at 2000 rpm for 10 minutes. During the deposition cycles the polymer solutions were maintained at 5° C. by cooling in a refrigerator.

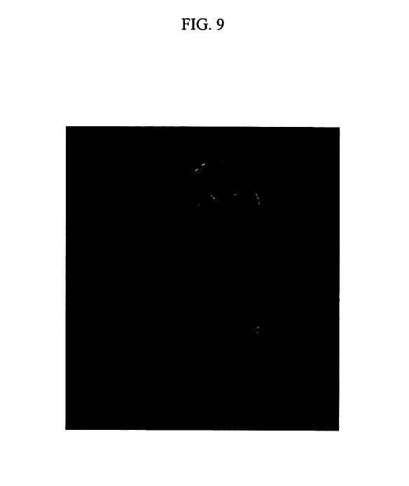

The thickness of a single wall of the resulting (polyethyleneimine/polymethacrylic acid)(polyvinylmethyl ether/polymethacrylic acid)₃ capsules was determined by electron energy-loss spectrometry and made up 52.4±3.7 nm. The typical fluorescence microscopy image of the capsules of the kind is shown in FIG. 9, which is a fluorescence microscopy image of (polyethyleneimine/polymethacrylic acid)(polyvinylmethyl ether/polymethacrylic acid)₃ capsules stained with Alexa Fluor 488 dihydrazide sodium salt fluorescent dye. The initial template was cadmium carbonate.

### 5.9.11 Preparation of Temperature-Responsive Capsules of Polyvinyl Caprolactam/Polymethacrylic Acid

Precursor layers of polyethyleneimine and polymethacrylic acid from 0.2 mg/ml solution at pH=3.5 were deposited onto positively or negatively charged cadmium carbonate particles (16-20 μm) with three washing cycles at pH=3.5 using the procedure described above.

Next, polyvinyl caprolactam/polymethacrylic acid layers (the polyvinyl caprolactam had a molecular weigh of 500,000, and was purchased from Polymer Source Inc., Canada) were self-assembled at pH=3.5 starting from polyvinyl caprolactam via according to the procedure described above. The deposition time was 15 minutes. Each deposition cycle was followed by removing excess polymer by washing with a buffer solution at pH=3.5. As part of the washing procedure, centrifugation of the suspensions at 1200 rpm for 0.5 min removed the supernatant.

After the desired number of layers was deposited, the carbonate core was dissolved using a 0.1 M HCl solution as described above to yield hollow capsules. The resulting hollow capsules were washed several times with the HCl solution to remove core-decomposition products. As part of the washing procedure, the supernatant was removed after centrifugation at 2000 rpm for 10 minutes. During the deposition cycles the polymer solutions were kept at 5° C.

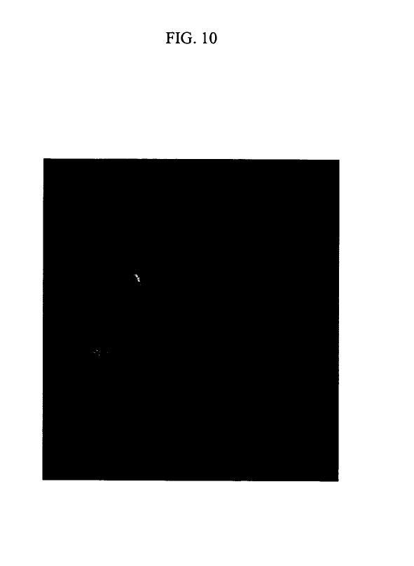

The thickness of a single wall of the resulting (polyethyleneimine/polymethacrylic acid)(polyvinyl caprolactam/polymethacrylic acid)₃ capsules was determined by Electron Energy-Loss Spectrometry and made up 51.0±11.1 nm. A fluorescence microscopy image of the capsules produced according to the above procedure is shown in FIG. 10, which is a fluorescence microscopy image of (polyethyleneimine/polymethacrylic acid)(polyvinyl caprolactam/polymethacrylic acid)₃ capsules stained with Alexa Fluor 488 dihydrazide sodium salt fluorescent dye. The initial template was cadmium carbonate.

### 5.9.12 Covalent Cross-Linking of Hydrogen-Bonded Multilayers via the Carboxylic Groups of Polymethacrylic Acid and the Functional Groups of a Difunctional Cross-Linking Reagent

As summarized in Table 1 below, (polymethacrylic acid/polyethyleneoxide)₅ multilayer or (poly-N-vinylpyrrolidone/polymethacrylic acid)₅ multilayers were self-assembled by adapting the procedure described in S. A. Sukhishvili et al., Layered, Erasable Polymer Multilayers Formed by Hydrogen-Bonded Sequential Self-Assembly, 35 MACROMOLECULES 301 (2002) according to the invention. The deposition was performed at pH=3.5.

Accordingly, as outlined in Scheme 1 below, the carboxylic groups of the self-assembled (polyethyleneoxide/polymethacrylic acid) or (poly-N-vinylpyrrolidone/polymethacrylic acid) were activated with a mixture of 5 mg/ml 1-ethyl-3-(3-dimethylamino-propyl)-carbodiimide hydrochloride (EDC) and 5 mg/ml N-hydroxysulfosuccinimide sodium salt solutions at pH 4.0 (for both poly-N-vinylpyrrolidone/polymethacrylic acid) multilayers and (polyethyleneoxide/polymethacrylic acid) multilayers.

Next, as outlined in Scheme 1 below, the layers were cross linked by reacting with 0.01 mg/ml of the difunctional cross linker ethylene diamine at the appropriate pH (see Table 1 below).

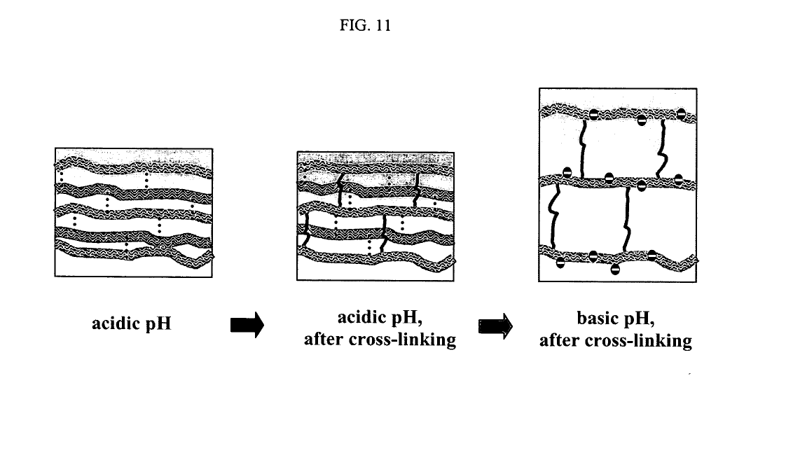

The results for each set of conditions are shown in Table 1 below. The resulting multilayer structure is schematically shown in FIG. 11. FIG. 11 schematically depicts the multilayer structure produced by covalent cross-linking of hydrogen-bonded multilayers via the carboxylic groups of polymethacrylic acid and the functional groups of a difunctional cross-linking reagent.

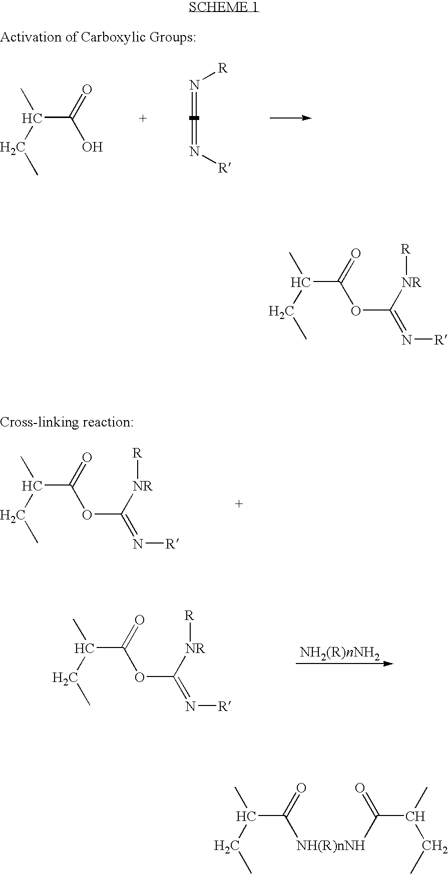

TABLE 1
CONDITIONS AND RESULTS

| | | Difunctionalized | Neutral | Percent |
|---|---|---|---|---|
| | Activation | cross-linker and pH | polymer | polymethacrylic |
| Neutral polymer | conditions | conditions | release, pH | acid cross-linked |
| (poly-N-vinyl-pyrrolidone/poly- | EDC | Ethylene diamine | 5-5.8 | pH 9 (90%) |
| (methacrylic acid))₅ 10 nm per bilayer | pH = 4 | pH 5.8 | | |
| (polyethyleneoxide/polymethacrylic | EDC | Ethylene diamine | 4-7 | pH 7 (50%) |
| acid)₅ 28 nm per a bilayer | pH = 4 | pH 4.3 | | |

### 5.9.13 Covalent Cross-Linking of Hydrogen-Bonded: the Carboxylic Groups of Polymethacrylic Acid and the Functional Groups of a Difunctionalized Poly(Ethylene Glycol)

As summarized in the tables below, (polymethacrylic acid/difunctionalized polyethylene glycol) multilayers were self-assembled by adapting the procedure described in S. A. Sukhishvili et al., Layered, Erasable Polymer Multilayers Formed by Hydrogen-Bonded Sequential Self-Assembly, 35 MACROMOLECULES 301 (2002) according to the invention using the difunctionalized polyethylene glycols shown in the table below. The deposition was performed at pH=3.5.

TABLE
DIFUNCTIONALIZED POLY(ETHYLENE GLYCOLS)

polyethyleneoxide-(NH₂)₂ - Poly (ethylene glycol), diamino terminated,
Mw 2,000
polyethyleneoxide-(hydrazide)₂ - Poly (ethylene glycol), dihydrazide
terminated, Mw 3,400
polyethyleneoxide-(COOH)₂ - poly (ethylene glycol), dicarboxymethyl
terminated, Mw 3,400

Next, the carboxylic groups of the self-assembled polymers were activated with a mixture of 5 mg/ml 1-ethyl-3-(3-dimethylaminopropyl)-carbodiimide hydrochloride and 5 mg/ml N-hydroxysulfosuccinimide sodium salt solutions at pH 4.0. Two hours were allowed to complete covalent cross-linking reaction between the activated carboxylic groups of polymethacrylic acid and the end groups of difunctionalized poly (ethylene glycol) layers within the multilayers.

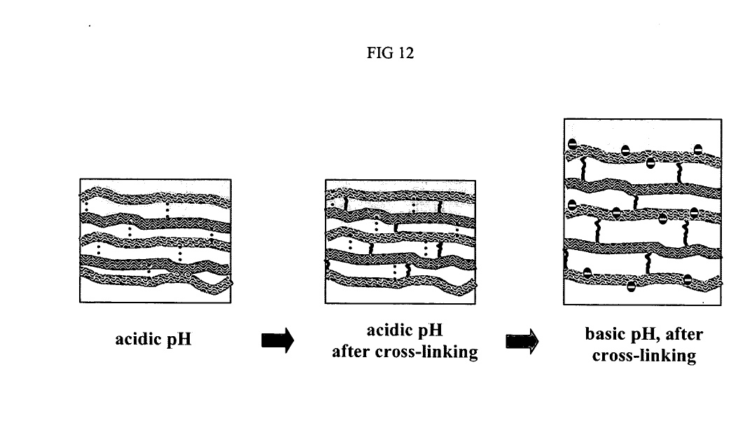

The stability ranges are shown in the table below. The resulting multilayer structure is schematically shown in FIG. 12.

TABLE
CONDITIONS AND RESULTS

| | | | | pH at which % |
|---|---|---|---|---|
| | | cross-linking | pH of neutral | polymethacrylic |
| Hydrogen-Bonded Systems | Activator | reagent | polymer release | acid released |
| (polymethacrylic acid/ | EDC | Ethylene | 4-7 | pH 7-45% |
| polyethyleneoxide-(NH₂)₂)₅ | | diamine | | |
| 16 nm per a bilayer | | | | |
| (polymethacrylic acid/ | EDC | Ethylene | 6.5 | pH 8-25% |
| polyethyleneoxide-(Hydrazide)₂)₅ | | diamine | | |
| 26 nm per a bilayer | | | | |
| (polymethacrylic acid/ | EDC & | Adipic acid | 8 | pH 8-50% |
| polyethyleneoxide-(Hydrazide)₂)₅ | NHSS | dihydrazide | | |
| 26 nm per a bilayer | | | | |
| (polymethacrylic acid/ | EDC & | Adipic acid | 7-9.6 | pH 9.6-5% |
| polyethyleneoxide-(COOH)₂)₃ | NHSS | dihydrazide | | |
| 22 nm per a bilayer | | | | |

5.10 Definitions

As used herein, the term neutral polymer means a polymer that has no ionic bonds, and is composed only of covalent bonds. That is, there are no ionized groups or salts present on the polymer and thus no charged groups.

### 5.11 Conclusion

In view of the above Background, Summary, Figures, and Detailed Description, it is clear that in certain embodiments, the invention relates to an article comprising:
(a) a particle;
(b) a first neutral polymer film; and
(c) a second neutral polymer film contacting the first neutral polymer film, wherein the particle is partly or substantially soluble in an aqueous medium.

In another embodiment, the invention relates to an article comprising:
(a) a particle;
(b) a first neutral polymer film; and
(c) a second neutral polymer film contacting the first neutral polymer film,
wherein the diameter of the particle is of from about 3.5 nm to about 3.5 mm.

In another embodiment, the invention relates to a capsule comprising:
(a) a first neutral polymer film;
(b) a second neutral polymer film contacting the first neutral polymer film, and
(c) a cavity.

In yet another embodiment, the invention relates to a method of making a capsule comprising:
(a) contacting a solution of a first uncharged polymer with a particle having a volume of from about 50 nm³ to about 50 mm³ to coat the particle with a first uncharged polymer film;
(b) contacting the coated particle with a solution a second uncharged polymer to coat the coated particle with a second uncharged polymer film.

In still yet another embodiment, the invention relates to a method of making a capsule comprising:
(a) contacting a solution of a first uncharged polymer with a particle to coat the particle with a first neutral polymer film;
(b) contacting the coated particle with a solution of a second uncharged polymer to coat the coated particle with a second neutral polymer film.

The present invention is not to be limited in scope by the specific embodiments disclosed in the description and examples, which are intended as illustrations of a few embodiments of the invention. Any embodiments that are functionally equivalent to those described above are within the scope of this invention. Indeed, various modifications of the invention in addition to those shown and described herein will become apparent to those skilled in the art and are intended to fall within the scope of the appended claims. All cited references are hereby incorporated herein in their entireties by reference.

1. An article comprising:
(a) a particle;
(b) a first neutral polymer film; and
(c) a second neutral polymer film contacting the first neutral polymer film,
wherein the particle is partly or substantially soluble in an aqueous medium.

2. The article of claim 1, wherein the aqueous medium comprises an inorganic or organic acid or an inorganic or organic base.

3. The article of claim 1, wherein the first neutral polymer film comprises a hydrogen bond donor.

4. The article of claim 3, wherein the first neutral polymer film comprises a polycarboxylic acid.

5. The article of claim 3, wherein the first neutral polymer film comprises a polymethacrylic acid, a polynucleotide, or a polymer of a vinyl nucleic acid or a copolymer thereof.

6. The article of claim 1, wherein the second neutral polymer film comprises a hydrogen-bond acceptor.

7. The article of claim 6, wherein the second neutral polymer film comprises a polyether, a polyketone, a polyaldehyde, a polyacrylamide, a polyamine, a polyester, a polyphosphazene, or a polysaccharide or a copolymer thereof.

8. The article of claim 6, wherein the second neutral polymer film comprises polyethylene oxide, poly-1,2-dimethoxyethylene, poly(vinylmethyl ether), poly(vinylbenzo-18-crown-6), polyvinyl butyral, poly(N-vinyl-2-pyrrolidone), polyacrylamide, polymethacrylamide, poly(N-isopropylacrylamide), poly(4-amine)styrene, poly(cylohexane-1,4-dimethylene terephthalate), polyhydroxy methyl acrylate, poly(bis(methylamino)phosphazene), poly(bis(methoxyethoxyethoxy)phosphazene, carboxymethyl cellulose or a copolymer thereof.

9. The article of claim 1, wherein the first neutral polymer film and the second neutral polymer film are crosslinked.

10. The article of claim 1, wherein the particle comprises a mineral, an inorganic salt, an organic compound, or a salt of an organic compound.

11. The article of claim 1, wherein the particle comprises calcium carbonate, cadmium carbonate or manganese carbonate.

12. The article of claim 1, comprising a biomaterial.

13. The article of claim 12, wherein the biomaterial comprises a cell or a genetic material.

14. The article of claim 1, comprising a bioactive agent or a pharmaceutical.

15. The article of claim 14, wherein the bioactive agent or pharmaceutical comprises a small-molecule drug, a vaccine, an antibody, a hormone, a growth factor, a sex sterilant, a fertility inhibitor, a fertility promoter, a protein, a peptide, a fragrance, a flavor, a vitamin, or a nutrient.

16. The article of claim 1, comprising a chemical agent.

17. The article of claim 16, wherein the chemical agent comprises a nucleoside, a nucleotide, an oligonucleoside, an oligonucleotide, an agricultural material, a fertilizer, a pesticide, a preservative, a catalyst, an enzyme, a polymer, a colorant, a dye, a fluorescent compound, a sensor molecule, an excipient, a surfactant, a detergent, or a chemical used in environmental remediation.

18. An article comprising:
(a) a particle;
(b) a first neutral polymer film; and
(c) a second neutral polymer film contacting the first neutral polymer film,
wherein the diameter of the particle is of from about 3.5 nm to about 3.5 mm.

19. The article of claim 18, wherein the diameter of the particle is of from about 15 nm to about 1 mm.

20. The article of claim 18, wherein the diameter of the particle is of from about 25 nm to about 40 μm.

21. The article of claim 18, wherein the diameter of the particle is of from about 40 nm to about 20 μm.

22. The article of claim 18, wherein the diameter of the particle is of from about 80 nm to about 10 μm.

23. The article of claim 18, wherein the first neutral polymer film comprises a hydrogen bond donor.

24. The article of claim 18, wherein the first neutral polymer film comprises a polycarboxylic acid.

25. The article of claim 24, wherein the first neutral polymer film comprises a polymethacrylic acid, a polynucleotide, or a polymer of a vinyl nucleic acid or a copolymer thereof.

26. The article of claim 18, wherein the second neutral polymer film comprises a hydrogen-bond acceptor.

27. The article of claim 26, wherein the second neutral polymer film comprises a polyether, a polyketone, a polyaldehyde, a polyacrylamide, a polyamine, a polyester, a polyphosphazene, or a polysaccharide or a copolymer thereof.

28. The article of claim 26, wherein the second neutral polymer film comprises polyethylene oxide, poly-1,2-dimethoxyethylene, poly(vinylmethyl ether), poly(vinylbenzo-18-crown-6), polyvinyl butyral, poly(N-vinyl-2-pyrrolidone), polyacrylamide, polymethacrylamide, poly(N-isopropylacrylamide), poly(4-amine)styrene, poly(cylohexane-1,4-dimethylene terephthalate), polyhydroxy methyl acrylate, poly(bis(methylamino)phosphazene), poly(bis(methoxyethoxyethoxy)phosphazene, carboxymethyl cellulose or a copolymer thereof.

29. The article of claim 18, wherein the first neutral polymer film and the second neutral polymer film are crosslinked.

30. The article of claim 18, wherein the particle comprises a mineral, an inorganic salt, an organic compound, or a salt of an organic compound.

31. The article of claim 18, wherein the particle comprises calcium carbonate, cadmium carbonate or manganese carbonate.

32. The article of claim 18, comprising a biomaterial.

33. The article of claim 32, wherein the biomaterial comprises a cell or a genetic material.

34. The article of claim 18, comprising a bioactive agent or a pharmaceutical.

35. The article of claim 34, wherein the bioactive agent or pharmaceutical comprises a small-molecule drug, a vaccine, an antibody, a hormone, a growth factor, a sex sterilant, a fertility inhibitor, a fertility promoter, a protein, a peptide, a fragrance, a flavor, a vitamin, or a nutrient.

36. The article of claim 18, comprising a chemical agent.

37. The article of claim 36, wherein the chemical agent comprises a nucleoside, a nucleotide, an oligonucleoside, an oligonucleotide, an agricultural material, a fertilizer, a pesticide, a preservative, a catalyst, an enzyme, a polymer, a colorant, a dye, a fluorescent compound, a sensor molecule, an excipient, a surfactant, a detergent, or a chemical used in environmental remediation.

38. A capsule comprising:
(a) a first neutral polymer film;
(b) a second neutral polymer film contacting the first neutral polymer film, and
(c) a cavity.

39. The capsule of claim 38, wherein the diameter of the cavity is of from about 3.5 nm to about 3.5 mm.

40. The capsule of claim 38, wherein the diameter of the cavity is of from about 15 nm to about 1 mm.

41. The capsule of claim 38, wherein the diameter of the cavity is of from about 25 nm to about 40 μm.

42. The capsule of claim 38, wherein the diameter of the cavity is of from about 40 nm to about 20 μm.

43. The capsule of claim 38, wherein the diameter of the cavity is of from about 80 nm to about 10 μm.

44. The capsule of claim 38, wherein the first neutral polymer film comprises a hydrogen bond donor.

45. The capsule of claim 44, wherein the first neutral polymer film comprises a polycarboxylic acid.

46. The capsule of claim 44, wherein the first neutral polymer film comprises a polymethacrylic acid, a polynucleotide, or a polymer of a vinyl nucleic acid or a copolymer thereof.

47. The capsule of claim 38, wherein the second neutral polymer film comprises a hydrogen-bond acceptor.

48. The capsule of claim 47, wherein the second neutral polymer film comprises a polyether, a polyketone, a polyaldehyde, a polyacrylamide, a polyamine, a polyester, a polyphosphazene, or a polysaccharide or a copolymer thereof.

49. The capsule of claim 47, wherein the second neutral polymer film comprises polyethylene oxide, poly-1,2-dimethoxyethylene, poly(vinylmethyl ether), poly(vinylbenzo-18-crown-6), polyvinyl butyral, poly(N-vinyl-2-pyrrolidone), polyacrylamide, polymethacrylamide, poly(N-isopropylacrylamide), poly(4-amine)styrene, poly(cylohexane-1,4-dimethylene terephthalate), polyhydroxy methyl acrylate, poly(bis(methylamino)phosphazene), poly(bis(methoxyethoxyethoxy)phosphazene, carboxymethyl cellulose or a copolymer thereof.

50. The capsule of claim 38, wherein the first neutral polymer film and the second neutral polymer film are crosslinked.

51. The capsule of claim 38, comprising a biomaterial.

52. The capsule of claim 51, wherein the biomaterial comprises a cell or a genetic material.

53. The capsule of claim 38, comprising a bioactive agent or a pharmaceutical.

54. The capsule of claim 53, wherein the bioactive agent or pharmaceutical comprises a small-molecule drug, a vaccine, an antibody, a hormone, a growth factor, a sex sterilant, a fertility inhibitor, a fertility promoter, a protein, a peptide, a fragrance, a flavor, a vitamin, or a nutrient.

55. The capsule of claim 38, comprising a chemical agent.

56. The capsule of claim 55, wherein the chemical agent comprises a nucleoside, a nucleotide, an oligonucleoside, an oligonucleotide, an agricultural material, a fertilizer, a pesticide, a preservative, a catalyst, an enzyme, a polymer, a colorant, a dye, a fluorescent compound, a sensor molecule, an excipient, a surfactant, a detergent, or a chemical used in environmental remediation.

57. A method of making a capsule comprising:
(a) contacting a solution of a first uncharged polymer with a particle to coat the particle with a first neutral polymer film;
(b) contacting the coated particle with a solution of a second uncharged polymer to coat the coated particle with a second neutral polymer film.

58. The method of claim 57, wherein the particle is partly or substantially soluble in an aqueous medium

59. The method of claim 57, wherein the diameter of the particle is of from about 3.5 nm to about 3.5 mm.

60. The method of claim 57, wherein the diameter of the particle is of from about 15 nm to about 1 mm.

61. The method of claim 57, wherein the diameter of the particle is of from about 25 nm to about 40 μm.

62. The method of claim 57, wherein the diameter of the particle is of from about 40 nm to about 20 μm.

63. The method of claim 57, wherein the diameter of the particle is of from about 80 nm to about 10 μm.

64. The method of claim 58, further comprising contacting the particle coated with a second neutral polymer film with the aqueous medium to form a cavity.

65. The method of claim 64, wherein the diameter of the cavity is of from about 3.5 nm to about 3.5 mm.

66. The method of claim 64, wherein the diameter of the cavity is of from about 15 nm to about 1 mm.

67. The method of claim 64, wherein the diameter of the cavity is of from about 25 nm to about 40 μm.

68. The method of claim 64, wherein the diameter of the cavity is of from about 40 nm to about 20 μm.

69. The method of claim 64, wherein the diameter of the cavity is of from about 80 nm to about 10 μm.

70. The method of claim 57, wherein the aqueous medium comprises an inorganic or organic acid or an inorganic or organic base.

71. The method of claim 57, wherein the first neutral polymer film comprises a hydrogen bond donor.

72. The method of claim 71, wherein the first neutral polymer film comprises a polycarboxylic acid.

73. The method of claim 71, wherein the first neutral polymer film comprises a polymethacrylic acid, a polynucleotide, or a polymer of a vinyl nucleic acid or a copolymer thereof.

74. The method of claim 57, wherein the second neutral polymer film comprises a hydrogen-bond acceptor.

75. The method of claim 74, wherein the second neutral polymer film comprises a polyether, a polyketone, a polyaldehyde, a polyacrylamide, a polyamine, a polyester, a polyphosphazene, or a polysaccharide or a copolymer thereof.

76. The method of claim 74, wherein the second neutral polymer film comprises polyethylene oxide, poly-1,2-dimethoxyethylene, poly(vinylmethyl ether), poly(vinylbenzo-18-crown-6), polyvinyl butyral, poly(N-vinyl-2-pyrrolidone), polyacrylamide, polymethacrylamide, poly(N-isopropylacrylamide), poly(4-amine)styrene, poly(cylohexane-1,4-dimethylene terephthalate), polyhydroxy methyl acrylate, poly(bis(methylamino)phosphazene), poly(bis(methoxyethoxyethoxy)phosphazene, carboxymethyl cellulose or a copolymer thereof.

77. The method of claim 57, wherein the first neutral polymer film and the second neutral polymer film are crosslinked.

78. The method of claim 57, further comprising incorporating a biomaterial into the capsule.

79. The method of claim 78, wherein the biomaterial comprises a cell or a genetic material.

80. The method of claim 57, further comprising incorporating a bioactive agent or a pharmaceutical into the capsule.

81. The method of claim 80, wherein the bioactive agent or pharmaceutical comprises a small-molecule drug, a vaccine, an antibody, a hormone, a growth factor, a sex sterilant, a fertility inhibitor, a fertility promoter, a protein, a peptide, a fragrance, a flavor, a vitamin, or a nutrient.

82. The method of claim 57, further comprising incorporating a chemical agent into the capsule.

83. The method of claim 82, wherein the chemical agent comprises a nucleoside, a nucleotide, an oligonucleoside, an oligonucleotide, an agricultural material, a fertilizer, a pesticide, a preservative, a catalyst, an enzyme, a polymer, a colorant, a dye, a fluorescent compound, a sensor molecule, an excipient, a surfactant, a detergent, or a chemical used in environmental remediation.
